(19) United States
(12) Patent Application Publication
(10) Pub. No.: US 2024/0065568 A1
(43) Pub. Date: Feb. 29, 2024

(54) ACOUSTEOMIC SENSING AND MONITORING USING A HUMAN-CENTRIC INTELLIGENT ACOUSTEOMIC ARRAY

(71) Applicant: THE JOHNS HOPKINS UNIVERSITY, Baltimore, MD (US)

(72) Inventors: Andreas G. ANDREOU, Baltimore, MD (US); Rajat MITTAL, Vienna, VA (US); Christos SAPSANIS, Patras (GR); Jung Hee SEO, Baltimore, MD (US); W. Reid THOMPSON, Baltimore, MD (US); Jon R. RESAR, Stevenson, MD (US)

(73) Assignee: THE JOHNS HOPKINS UNIVERSITY, Baltimore, MD (US)

(21) Appl. No.: 18/259,345

(22) PCT Filed: Dec. 23, 2021
(86) PCT No.: PCT/US2021/065043

§ 371 Date: Jun. 26, 2023

Related U.S. Application Data
(60) Provisional application No. 63/132,188, filed on Dec. 30, 2020.

(51) Int. Cl. A61B 5/026; A61B 5/00; A61B 5/282; G06T 7/00

(52) U.S. Cl. CPC A61B 5/026; A61B 5/282; A61B 5/6805; G06T 7/0012; A61B 2562/0204; G06T 2207/20081; G06T 2207/30048

(57) ABSTRACT

An appliance for monitoring blood flow is provided. The appliance includes a plurality of spatially separated acousteomic sensors for auscultation detection of a patient; a hardware processor and a non-transitory computer-readable medium that stores a trained computer model for modeling a function of a healthy heart for analyzing the acousteomic signals; and a transmitter that transmits the acousteomic signals from the plurality of acousteomic sensors.

This application claims priority to U.S. Provisional Patent Application Ser. No. 63/132,188 filed Dec. 30, 2020, the disclosure of which is incorporated herein by reference in its entirety.

### GOVERNMENT RIGHTS

This invention was made with Government support under grants IIS-1344772 and SMA-1540916 awarded by the National Science Foundation. The Government has certain rights in this invention.

### FIELD

The present teachings generally relate acousteomic sensing and monitoring using a human-centric intelligent acousteomic array.

### BACKGROUND

Cardiovascular disease (CVD) is a class of diseases and disorders of the heart and blood vessels, which can include coronary heart disease, cerebrovascular disease, rheumatic heart disease, chronic and acute valve failures and other conditions. In the industrialized world current trends in aging, obesity and diabetes point to a world-wide worsening outlook for CVD diseases.

In the U.S.A, the national annual expenditure on heart disease exceeds half a trillion with over half a million deaths attributed to CVD7. For example, over 6 million adults in the U.S. and Europe have some form of aortic valve disease (1.8%); for adults over 60 years old the prevalence is 10.7%. Of these, ˜1 million have severe aortic stenosis (AS), though only ˜50% of these are symptomatic. Without treatment of severe AS, ˜50% will die within 2 years after onset of symptoms. ˜100K aortic valve procedures are performed in the U. S.A every year7. In children, valvar aortic stenosis is one of the most common congenital defects. Though relatively rare to present in infancy (˜2% of infants with critical heart disease), in older children and adolescents, it represents an increasing percentage of heart disease, second only to ventricular septal defect by the third decade of life. Bicuspid aortic valve (2 aortic valve leaflets instead of 3) is present in 1-2% of the population and accounts for a large proportion of aortic valve disease in children and adults, and can lead to or be associated with valvar stenosis or regurgitation, endocarditis, or ascending aortic aneurysm.

The use of cardiac prostheses is growing rapidly. With a population that is aging rapidly, the rate of deployment of cardiac, cardiovascular and cerebrovascular prostheses and implants such as heart valves, embolization devices, vascular stents, annuloplasty rings, and ventricular assist devices has grown rapidly. This trend has been accelerated by the development of innovative endovascular and transcatheter systems that can deploy prostheses that would previously have required highly invasive surgery. The overall consequence of this trend is a rapidly growing population of patients who are (or will be) living with cardiac prostheses. Until about 2011, aortic valve replacement required a highly invasive open-heart surgery, a complex, costly and risky procedure. Transcatheter aortic valve (TAV) replacement can be used for replacement of dysfunctional native aortic valves via a transcatheter procedure in the catherization laboratory in about one hour with most patients discharged in 24 hrs. TAVs are being deployed at the rate of 125K/year worldwide, a rate expected to double in about 5 years.

Proactive monitoring of a cardiac prostheses is essential but lacking convenient means to do so. Cardiac prostheses are particularly prone to various “malfunctions” because they are implanted in an organ that undergoes constant movement/deformation and they are exposed to the dynamic flow of blood and its constituents. For instance, a TAV implant can experience early leaflet thrombosis, infective endocarditis, paravalvular leaks, leaflet tears and stent deformation. Early leaflet thrombosis in particular, has emerged as a serious and persistent issue. This condition may remain asymptomatic, but then lead to an acute event such as embolic stroke. On the positive side, if detected early, it can be easily managed with anticoagulation therapy. However, detection of early leaflet thrombosis requires the use of transesophageal echo, CT or MR. Such monitoring is (a) expensive (b) invasive, (c) disruptive for the patients; and (d) not a cost-effective use of the limited resources of a hospital. Thus, while proactive monitoring of such cardiac prostheses is essential, it is also lacking in the current, hospital-centric standard-of-care regime.

Today, auscultation remains the primary method for CVD screening, including aortic valve disease, despite multiple reports of declining auscultation proficiency in most primary care trainees. For example, of particular difficulty for even the trained clinician is the recognition of the early systolic ejection click. The harsh systolic ejection murmur of Aortic Stenosis (AS) occurs as flow across the valve in systole is obstructed by a smaller than normal effective orifice, creating a pressure differential between the ventricle and ascending aorta and has a characteristic wide band frequency content (“harsh” quality murmur) that radiates from the right upper sternal border into the neck. In cases of preserved ventricular function, increasing severity of stenosis results in increasing intensity of the murmur. Aortic Regurgitation (AR) occurs in early diastole, when the aortic valve is closed, and has a characteristic blowing, “decrescendo” murmur, the length of which is related to severity of the AR, and the Left Ventricle (LV) versus systemic arterial diastolic pressures.

Nowadays, severe CVD patients can be accurately monitored only in the ICU or in CIC. Virtually every heart condition has a distinct acousteomic signature which can be traced to abnormal hemodynamics localized somewhere in the cardiovascular system. Heart sounds from an individual contain significant amounts of disease related information, but cardiac auscultation currently uses only a small fraction of this information for diagnosis as it lacks the ability to create a longitudinal archive of disease signatures. Furthermore, as indicated earlier cardiac auscultation is a skill in decline because newer generations of cardiologist and physicians are not being trained properly in the art and science of cardiac auscultation. Thus, a valuable diagnostic modality is therefore falling into disuse and it is in dire need of new insights and innovative new technology-based solutions. Additionally, as evident from recent studies, cardiac auscultation is a process that has implicit gender, physique and race biases introduced by the state of mind of the doctor or medic. Hence, new approaches to heart-disease management that are effective as well as inexpensive are needed.

COVID-19 pandemic is challenging modern medicine and public health delivery models bringing telemedicine and e-Health in the spotlight. For over two months, the SARS-2-CoV-2 coronavirus is ravaging the world resulting in large numbers of human casualties and it is stressing hospitals and clinics, while most non COVID-19 patients have been unable or unwilling to seek help from professionals in person. Information technology has advanced phenomenally and in the years to come, medical practice over the Internet is poised to become part of routine medical care. Alas! Effective telemedicine and e-Health, necessitates timely and cost-effective acquisition of essential vital information from patients. The latter is currently the weak link at what can be done remotely. For example, even the simplest medical diagnosis process of auscultation, i.e. the action of listening to sounds from a patient with a stethoscope is problematic in a remote setting.

### SUMMARY

In accordance with examples of the present disclosure, an appliance for monitoring the state of a cardiovascular system is disclosed. The appliance comprises a plurality of spatially separated acousteomic sensors for auscultation detection of a patient; a hardware processor and a non-transitory computer-readable medium that stores a trained computer model for modeling a function of a healthy heart for analyzing the acousteomic signals; and a transmitter that transmits the acousteomic signals from the plurality of acousteomic sensors.

Various additional features of the appliance can include one or more the following features. The appliance can further comprise one or more electrocardiogram sensors that detect electrical signals produced by a heart. Embedded machine intelligence based on internal models can further analyze the electrical signals. The trained machine intelligence model can be trained using a physics-based virtual heart computer model that mimics the physical and physiological functioning of the heart. The analyzing can comprise comparing the acousteomic signals from the plurality of acousteomic sensors with a baseline of known healthy acousteomic signals from the trained computer model. The analyzing can comprise comparing the acousteomic signals from the plurality of acousteomic sensors and the electrical signals with a baseline of known healthy acousteomic signals and known healthy electrical signals from the trained machine intelligence model. The analyzing can comprise determining an abnormality in at least one of the plurality of the acousteomic signals, at least one of the electrical signals, or both, based on the comparing. The abnormality can comprises a thrombosis, a malfunction of an artificial valve, or both. The plurality of acousteomic sensors can be part of a fabric that is physical contact with the patient.

In accordance with examples of the present disclosure, a system for monitoring cardio-vascular system is disclosed. The system can comprise a wearable garment comprising a plurality of spatially separated acousteomic sensors for auscultation detection of a patient and one or more electrocardiogram sensors that detect electrical signals produced by a heart of the patient; a hardware processor and a non-transitory computer-readable medium that stores a trained machine intelligence model that captures a function of a healthy heart for analyzing the acousteomic signals and the electrical signals; and a transmitter that transmits the acousteomic signals and the electrical signals that are analyzed. For example, in one or more aspects, the system does not require a person to physically move the sensor (stethoscope) and probe at different locations like traditional auscultation, but it relies on embedded intelligent signal/information processing algorithms and machine intelligence to focus the “listening process”

Various additional features of the system can include one or more the following features. The analyzing can comprise comparing the acousteomic signals from the plurality of acousteomic sensors and the electrical signals with a baseline of known healthy acousteomic signals and known healthy electrical signals from the trained computer model. The analyzing can comprise determining an abnormality in at least one of the plurality of the acousteomic signals, at least one of the electrical signals, or both, based on the comparing. The abnormality can comprise a thrombosis, a malfunction of an artificial valve, or both. The trained machine intelligence model can be trained using a physics-based virtual heart computer model that mimics the physical and physiological functioning of the heart.

In accordance with examples of the present disclosure, a computer-implemented method for cardiovascular system and blood flow is provided. The computing machinery implemented method can comprise detecting auscultation using a plurality of spatially separated acousteomic sensors for a patient; analyzing the acousteomic sensors using a hardware processor and a non-transitory computer-readable medium that stores a trained machine intelligence model that embodies the function of a healthy heart; and transmitting the acousteomic signals from the plurality of acousteomic sensors.

Various additional features of the system can include one or more of the following features. The computing machinery method can further comprise detecting electrical signals using one or more electrocardiogram sensors that detect electrical signals produced by a heart. The trained machine intelligence model can further analyze the electrical signals. The trained computer model can be trained using a physics-based virtual heart computer model that mimics the physical and physiological functioning of the heart.

### BRIEF DESCRIPTION OF THE DRAWINGS

The accompanying drawings, which are incorporated in, and constitute a part of this specification, illustrate implementations of the present teachings and, together with the description, serve to explain the principles of the disclosure. In the figures:

FIGS. 1A-1E show a wearable phonocardiographic (PCG) system 100 that is designed to record sounds emitted from the heart from multiple areas of the Chest simultaneously, according to examples of the present disclosure. In particular, FIGS. 1A and 1B show a readout board and power supply, respectively, FIG. 1C shows a calibrated acousteomic sensing module, FIG. 1D shows a vest and full system, and FIG. 1E shows nominal sensor location.

FIG. 2 shows an example of a TAV and CT scan of a TAV with leaflet thrombosis.

FIG. 3A show pilot data including longitudinal recording of temporal sound and EKG signals for a TAVR patient and FIG. 3B shows 3D acousteomic “maps” generated by the multisensory array.

FIG. 4 shows an automated auscultation-based TAV at home monitoring system with an acousteomic sensor array, according to examples of the present disclosure.

FIG. 5 shows a diagram depicting data and information flow for a physics-assisted machine intelligence algorithm where statistical models for signal analysis and inference receive input from sensors (low dimensional data) and high dimensional data from computational modeling using MTMS software are reduced to signal and inference models that in turn feed the ML/inference module for robust recognition performance.

FIG. 6 shows a system level design of the disclosed system, according to examples of the present disclosure. Only two channels are shown here but it is understood that it involves a plurality of channels.

FIG. 7 shows a schematic of MTMS, according to examples of the present disclosure.

FIG. 8 shows hypothesized components of the acousteomic signature from TAV implanted in a patient based on cardiologist experience and our pilot data.

FIGS. 9A-9C show that the temporal variation of the modal amplitudes represents the longitudinal change of the acousteomic signature.

FIG. 10 shows male and female thorax anatomies derived from the ViP (Virtual Population) models.

FIG. 11 shows a schematic scientific approach using modeling to determine bias due to body habitus and gender

FIG. 12 shows two types of sensors for cardiac auscultation and acousteomic sensing: acousteomic (left) and vibration (right).

FIGS. 13A-13C shows modeling of leaflet thrombosis on valve sounds. Left: Schematic of leaflet thrombosis and resulting reduced leaflet motion. Right: Preliminary results from MTMS for a normal and leaflet thrombosis valves. FIG. 13A shows FSI simulation results showing the velocity contours at peak systole. FIG. 13B shows time signal of simulated heart sounds from normal and thrombosed valves. FIG.13C shows linear discriminant analysis for projection of PCA modes performed with 8 simulation cases for various thrombosis severities.

FIG. 14 shows a schematic diagram of the LSTM node architecture is shown (top) with governing equations on the (bottom).

FIG. 15 is an example of a hardware configuration for a acousteomic processor, which can be used to perform one or more of the processes described above.

### DETAILED DESCRIPTION

Given the above complexities of hospital-based monitoring, monitoring at home emerges as a viable option for the population of patients in need of such medical service. Automated auscultation-based monitoring provides one sensing modality for detecting heart sounds/murmurs. For instance, the intensity, timing and frequency content of systolic ejection sounds from native aortic valves (“aortic ejection click”) is directly related to the dynamics of the valve and is a function of the stiffness and mass of the valve leaflets, and the frequency of valve vibrations. Similarly, the “S2” sounds associated with valve closure also contain signatures of the movement and dynamics of the native aortic valve. In addition, valve dysfunction usually results in stenosis, regurgitation or both, that in turn yields systolic or diastolic heart murmurs. Thus, heart sound is the only non-invasive modality that can provide effective monitoring of function of cardiac prostheses such as TAVs. Furthermore, persistent monitoring at home results in longitudinal data with unprecedented value that is amenable to sophisticated statistical analysis and models for predicting health of patients with these prostheses. The disclosed system does not require a person to physically move the sensor (stethoscope) and probe at different locations like traditional auscultation, but it relies on embedded intelligent signal/information processing algorithms and machine intelligence to focus the “listening process,”

Generally speaking, examples of the present disclosure provides for a semi-autonomous “robotic” cardiac auscultation, as well as other measurements such as electrocardiogram (ECG or EKG) that can provide direct information for impending acute cardiac events. The disclosed devices, systems, and methods leverages new capabilities in sensor technologies, computational modeling, smart signal processing and machine intelligence. Thus, resulting in diagnostic modalities that move away from management of heart conditions that today is mostly reactive, expensive and hospital-centric, and towards an approach that is smart, proactive, patient-centric and cost-effective. The disclosed devices and system can provide for some degree of operational autonomy by addition of wireless connectivity and augmentation with embedded machine intelligence and in signal and information processing algorithms running on energy aware hardware for optimal signal acquisition, processing and communication.

The disclosed devices, systems, and method provide for automated cardiac auscultation using acousteomic arrays that are sensitive to sounds that have frequencies in the human hearing range and beyond and are non-invasive and inexpensive, and can be used on a variety of medial modalities including, but are not limited to: (i) screening for particular heart conditions; (ii) longitudinal (tracking over time) assessment of cardiac health; (iii) 24/7, continuous, at-home health monitoring; and (iv) cardiac health assessment in rural and underdeveloped areas where access to specialists is limited. Additionally, hospital-centered modalities such as cardiac magnetic resonance imaging (MRI), computerized tomography (CT) and/or ECG can be used.

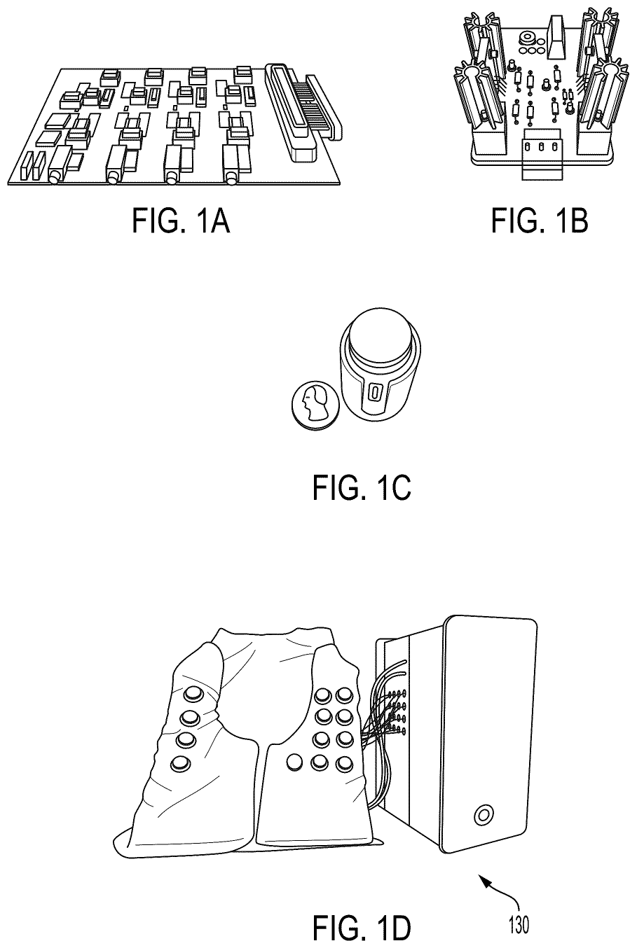

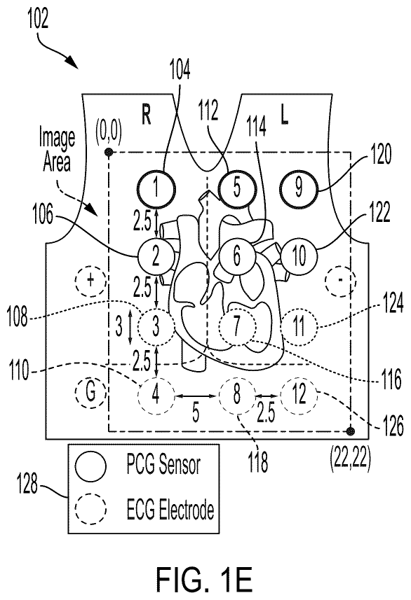

FIGS. 1A-1E show a wearable phonocardiographic (PCG) system 100 that is designed to record sounds emitted from the heart from multiple areas of the chest simultaneously, according to examples of the present disclosure. In particular, FIGS. 1A and 1B show a readout board and power supply, respectively, FIG. 1C shows a calibrated acousteomic sensing module, FIG. 1D shows a vest and full system, and FIG. 1E shows sensor location. In the example of FIGS. 1A-1E, the wearable phonocardiographic (PCG) system 100 is in the form of a vest that provides for automated cardiac auscultation.

The wearable phonocardiographic (PCG) system 100 comprises one or more PCG sensor arrays 102, where each PCG sensor array 102 comprises individual PCG sensor nodes 104 (1), 106 (2), 108 (3), 110 (4), 112 (3), 114 (6), 116 (7), 118 (8), 120 (9), 122 (10), 124 (11), and 126 (12) that can be arrayed within an inside lining of the vest. The individual PCG sensor nodes 104 (1), 106 (2), 108 (3), 110 (4), 112 (5), 114 (6), 116 (7), 118 (8), 120 (9), 122 (10), 124 (11), and 126 (12) can be connected to a readout system. The individual PCG sensor nodes 104 (1), 106 (21, 108 (3), 110 (4), 112 (5), 114 (6), 116 (7), 118 (8), 120 (9), 122 (10), 124 (11), and 126 (12) record the patient's heart and/or lung sounds in various regions of the chest around the heart. The individual PCG sensor nodes 101 (1), 106 (2), 108 (3), 110 (4), 112 (5), 114 (6), 116 (7), 118 (8), 120 (9), 122 (10), 124 (11), and 126 (12) are configured to be sensitive to acousteornic signals in at least the human audible range of about 20 Hz to about 20 kHz, and beyond that range.

The wearable phonocardiographic (PCG) system 100 can comprise a full readout system, and all the electronics, to be embedded within the vest and for the results to be sent to the patient's phone, making it portable, wireless and user friendly. The wearable phonocardiographic (PCG) system 100 allows for operational autonomy by the use of wireless connectivity and augmentation with embedded machine intelligence in signal and information processing algorithms running on energy aware hardware for optimal signal acquisition, processing and communication.

in some example, the wearable phonocardiographic (PCG) system 100 comprises one or more ECG electrodes 126.

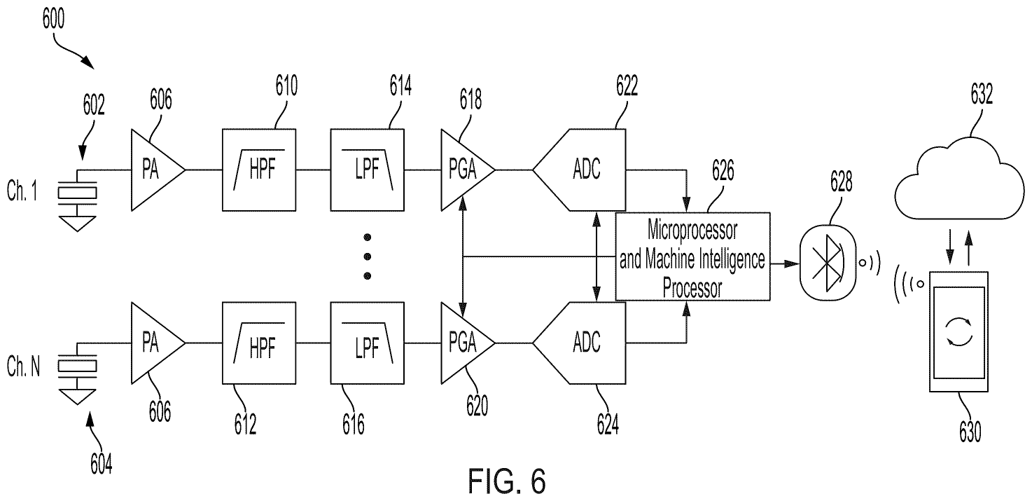

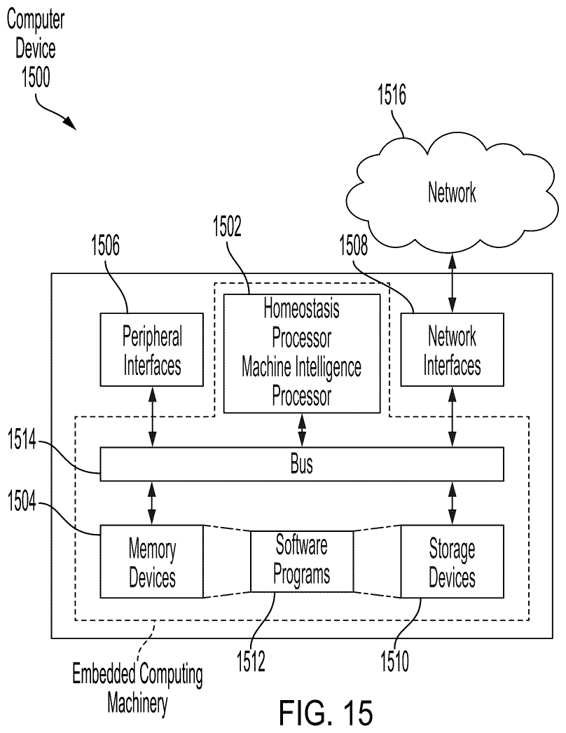

The wearable phonocardiographic (PCG) system 100 is configured to perform operations including denoising, localizing and separating acousteomic broadband sources in space by measuring spatial and temporal derivatives of the acousteomic field. Acousteomic and ECG information are gathered by the individual PCG sensor nodes 104 (1), 106 (2), 108 (3), 110 (4), 112 (5), 114 (6), 116 (7), 118 (8), 120 (9), 122 (10), 124 (11), and 126 (12) and the one or more ECG electrodes 128 on the garment that are connected to controller 130 for signal amplification, filtering and analog to digital conversion. A cable, such as a USB cable, can be used to connect controller 128 to computing machinery, such as shown in FIG. 6 and FIG. 15, for signal storage and analysis. Alternatively, the storage and analysis can be performed on an embedded in the vest computing machinery unit.

The measurements taken by the wearable PCG system 100 allow acquisition of both EKG and simultaneous sound and vibration recordings at a plurality of locations, such as the twelve shown in FIG. 1, on the chest and the synthesis of “acousteomic maps” that provide the ability for separating task relevant signals and not task relevant signals (often called noise), localization, failure tolerance and adaptation for body habitus and gender. The multisensory data also enables the use of advanced signal processing techniques.

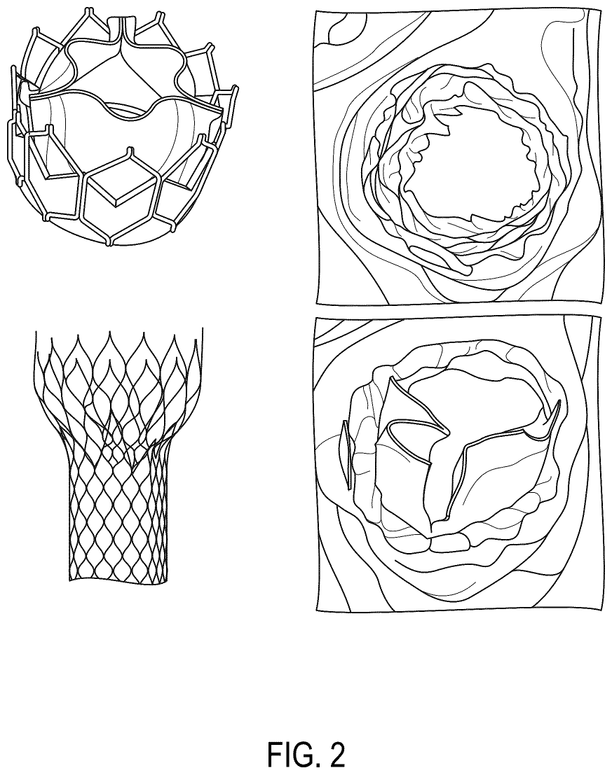

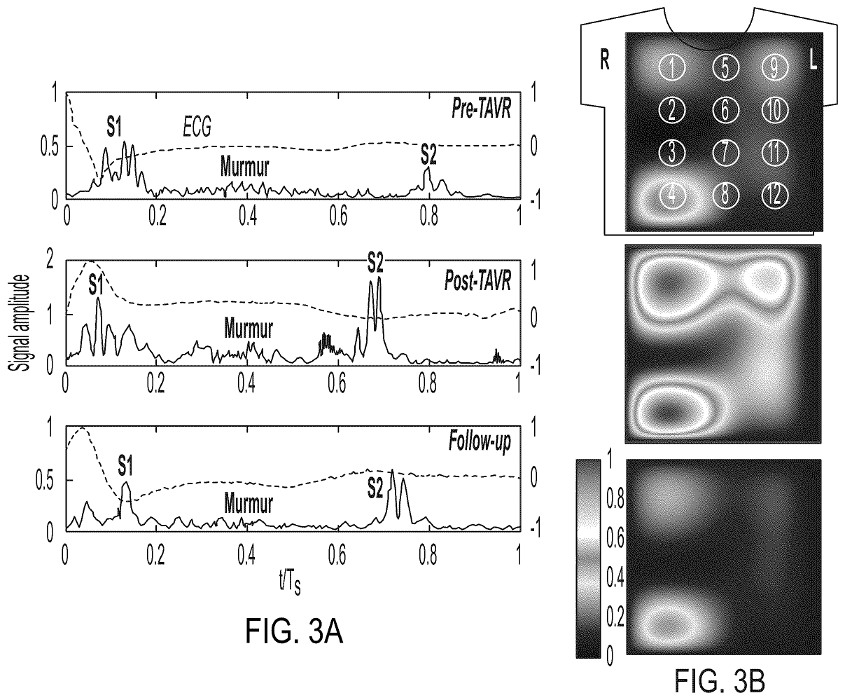

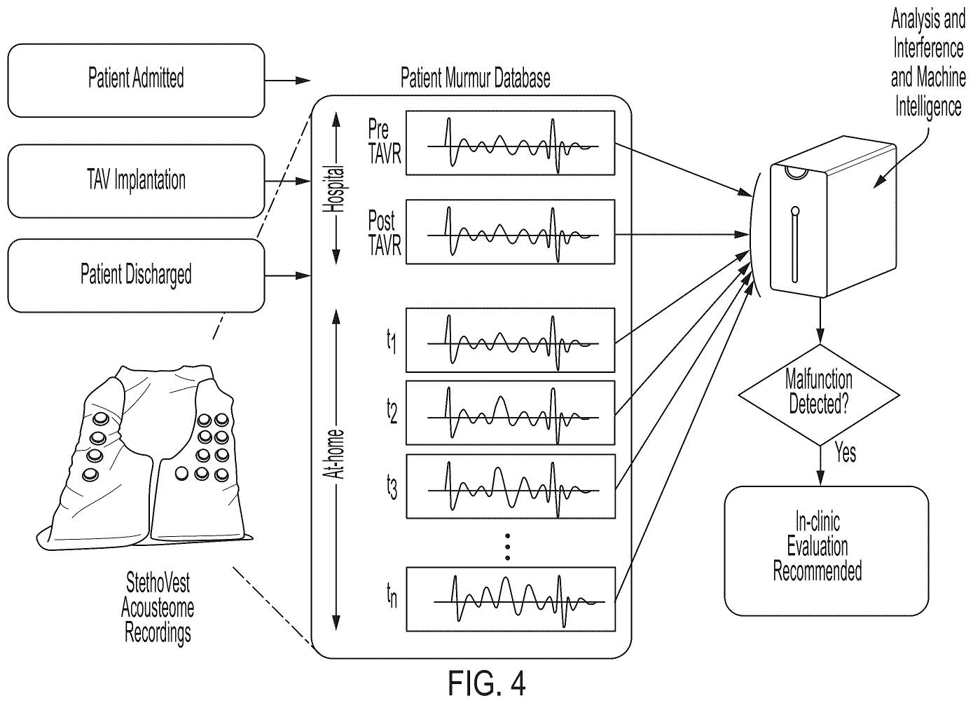

FIG. 2 shows an example of a TAV and CT scan of a TAV with leaflet thrombosis. FIG. 3A show pilot data including longitudinal recording of temporal sound and EKG signals for a TAVR patient and FIG. 3B shows 3D acousteomic “maps” generated by the multisensory array. FIG. 4 shows an automated auscultation-based TAV at home monitoring system with an acousteomic sensor array, according to examples of the present disclosure. The longitudinal data in FIG. 3A shows measurable differences in the sound signatures, which can be “mined” to detect valve dysfunction. This pilot data along with data from computer simulations provides evidence regarding the viability of automated auscultation for monitoring at home for example of prostheses like TAVs or for lung malfunction like COVID-19 and pneumonia.

FIG. 3 shows an automated auscultation-based TAV at home monitoring system with an acousteomic sensor array, according to examples of the present disclosure. The automated auscultation-based TAV at home monitoring system with an acousteomic sensor array can be augmented using one or more of: patient measurements, biomechanical models, or data-driven un-supervised learning techniques to characterize the longitudinal acousteomic signatures of implanted TAVs. Additionally, the automated auscultation-based TAV at home monitoring system with an acousteomic sensor array can employ in-silico virtual populations to quantify diagnostic “bias” in the measurements due to body habitus and gender and compensate for this bias via appropriate signal analysis and optimization of sensor design, placement and configuration. Further, the automated auscultation-based TAV at home monitoring system with an acousteomic sensor array can leverage in-silico biomechanical models of thrombosed valves to augment patient measurement, thereby enabling the development of physics-based machine intelligence inference models for robust detection and prediction of valve dysfunction. Furthermore, the data collected by the automated auscultation-based TAV at home monitoring system with an acousteomic sensor array can be represented as information-rich, spatio-temporal acousteomic maps of cardiac sounds that can be analyzed using machine intelligence based signal analysis to perform pattern analysis and machine intelligence to detect valve dysfunction via automated auscultation, as well as, proactively detect incipient prosthesis deterioration in a large and growing population of heart patients with cardiac valve implants.

The data collected by the automated auscultation-based TAV at home monitoring system with an acousteomic sensor array can be completed in less than ten minutes thereby minimizing inconvenience for the patient. The automated auscultation-based TAV at home monitoring system with an acousteomic sensor array allows for simultaneous variable placement of sensors, multisite and multimodal (PCG and ECG) recordings that provides redundancy to overcome loss or sub-optimality of signal from any sensor, the generation of four dimensional (two dimensions in space, time and frequency) maps of heart sound/vibrations patterns as well as the associated ECG signal, that can be used for source localization and identification, and multisite recordings that provide for sensor optimization and the use of signal features which are less affected by body habitus. The automated auscultation-based TAV at home monitoring system with an acousteomic sensor array can provide for wireless connectivity using one or more wireless technology, such as Bluetooth, that can be connected to a smart phone or similar type device.

The data acquired by the automated auscultation-based TAV at home monitoring system with an acousteomic sensor array can be used in biomechanical analysis based on “virtual populations,” and physics-based models that inform signal and inference/machine-learning algorithms for robust malfunction detection in TAVs. The physics-based models can employ data from Cardiac Auscultatory Recording Database (CARD), which contains patient historical and general physical examination data, electrocardiographic images and (ECG) diagnoses, echocardiographic diagnoses, and auscultatory findings made by the clinician using the traditional stethoscope.

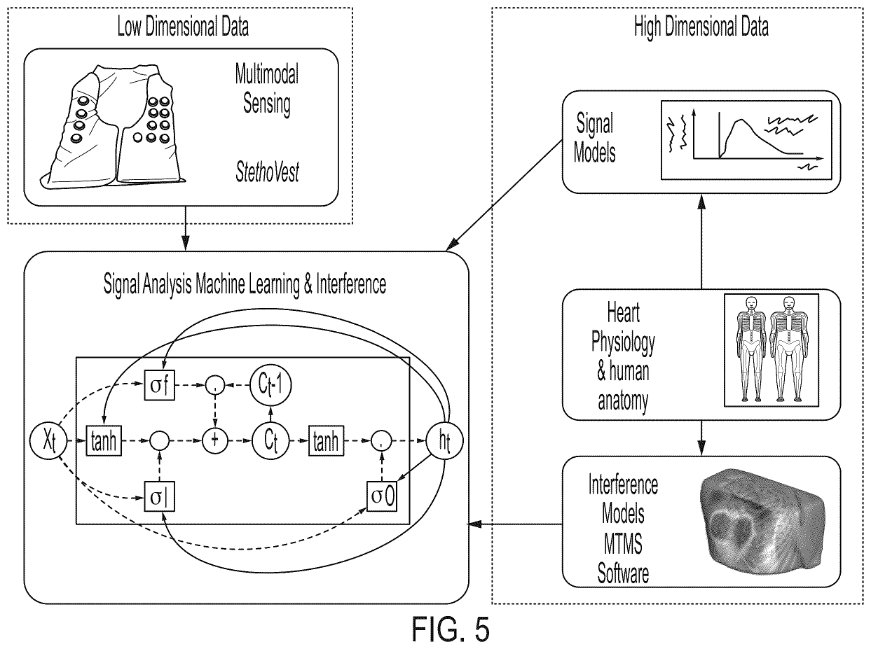

FIG. 5 shows low dimensional data generated from the array of multimodal sensors on the disclosed system to feed a physic-assisted machine intelligence algorithm and inference. The statistical models for signal analysis and inference receive input from sensors (low dimensional data). High dimensional data from computational modeling using MTMS software are reduced to signal and inference models that in turn feed to the ML/inference module for robust recognition performance.

FIG. 5 employs low dimensional data generated from the array of multimodal sensors on the disclosed system to feed machine intelligence (ML) and machine intelligence (MI) and inference. Complementary to the sensor signals, the ML/inference MI (machine intelligence) module is also provided with low dimensional signals from computational models that are derived from high dimensional complex phenomena of the underlying anatomy and physics of the heart. Deep neural networks and specifically the long short-term memory (LSTM) recurrent neural networks can be used to perform robust inference on temporal data by capturing important effects that originate in unknown underlying physical phenomena. A physics model-assisted approach can be used for pattern analysis and machine intelligence where there is paucity of data to train sophisticated deep neural network models with large number of parameters such as the LSTM.

The acousteomic array of the disclosed systemfor heart sound measurements can be augmented with a Multiphysics TAV Murmur Simulator (MTMS) which complement in-vivo studies via a silico computational models and physics-augmented model-based signal processing and machine-learning/inference tools. Complementary to the sensor signals, the ML/inference module is also provided with low dimensional signals from computational models that are derived from high dimensional complex phenomena of the underlying anatomy and physics of the heart. Deep neural networks and specifically the long short term memory (LSTM) recurrent neural networks have recently been shown to successfully perform robust inference on temporal data, by capturing important effects that originate in unknown underlying physical phenomena.

FIG. 6 shows a system level design 600 of the disclosed system, according to examples of the present disclosure. The system level design 600 provides for reduced power consumption, lower cost, noise sensitivity, as well as wireless connectivity and information assurance (secure collection and transmission). As shown in FIG. 6, each signal detected by each PCG sensor of the one or more individual PCG sensor nodes 104 (1), 106 (2), 108 (3), 110 (4), 112 (5), 114 (6), 116 (7), 118 (8), 120 (9), 122 (10), 124 (11), and 126 (12) can be provided on a separate communication channel. such as channel 1 602 and channel N 604, and amplified by respective amplifiers 606, 608 and processed using respective high passfi hers (HPFs) 610, 612, low pass filters (LPFs) 614, 616 amplified by respective programmable gain amplifiers (PGAs) 618,620, digitized by respective analog-to-digital converters (ADC) 622, 624, processed by controller 626, such as a rnicrocontroller or microprocessor and a machine intelligence processor, and wirelessly transmitted by wireless transceiver 628, such as using Bluetooth or other similar near field wireless protocols. Controller 626 is configured to control at least PDAs 618, 620, ADCs 622, 624. The data can be stored and transferred from a local memory, which acts as a buffer before the wireless transmission and/or saved locally in an on-board Secure Digital (SD) card for continuous monitoring. Portable computing device 630, such as a laptop, tablet, smartphone, or smartwatch, etc, can commutate wirelessly with wireless transceiver 628, which can wirelessly communicate with a network device or service 632, such as a cloud-base device or service.

A database of patient data can be collected that can be used to train a computing machinery and intelligence model. For example, 4-5 measurements can be made for each patient over the time-duration of 12-16 months resulting in a database of 1200+individual recordings and acousteome maps. Given the 10-15% incidence rate of TAV malfunctions, the model can tend to have around 40 cases with TAV malfunction within a sample cohort of about 320 patients. These data will be partitioned appropriately for robust training, validation, optimization and testing segments of the project.

Table 1 shows an example timeline of in-hospital measurements for TAVR patients that can be used to build the computer model. This timeline is based on the standard-of-care for TAVR patients and t₀ is the time of TAVR procedure.

| | |
|---|---|
| Pre TAVR | t₀−(6 ± 6) hrs. |
| Post TAVR | t₀−+(12 ± 12) hrs. |
| 1^{st} Follow-up | t₀−+(30 ± 10) hrs. |
| 2^{nd} Follow-up | t₀−+(12 ± 1) mos. |
| Additional | t₀−+(6 ± 6) mos. |

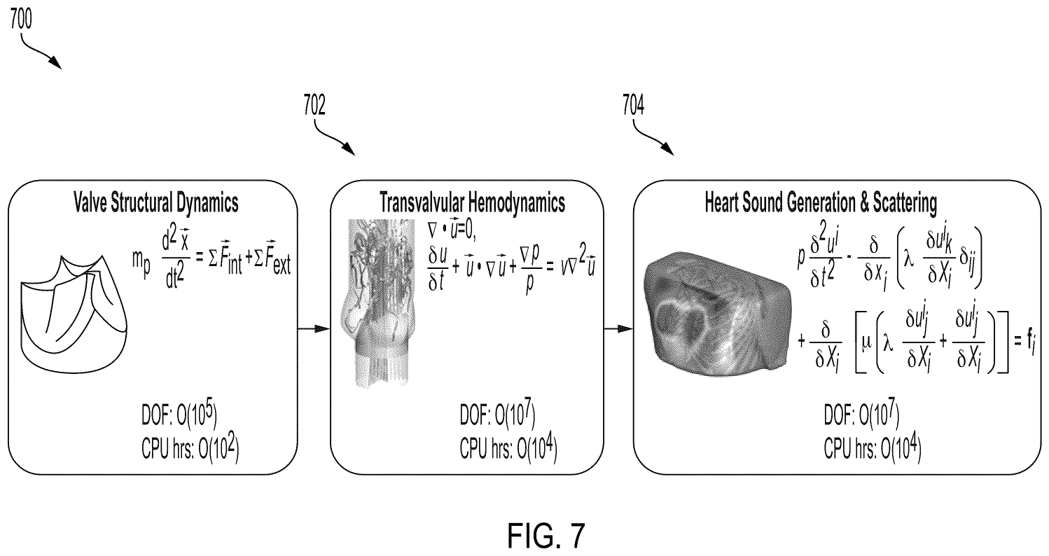

A multiphysics TAV murmur simulator (MTMS) can be used to investigate the effects of various TAV malfunctions on the heart sound and to generate a high-dimensional in-silico database for the valve sound analysis and machine intelligence models that can represent and/or augment the data in the computer model. FIG. 7 shows a schematic of MTMS 700, according to examples of the present disclosure. The MTMS 700 comprises fluid-structure-interaction (FSI) model 702 for resolving valve dynamics and blood flow dynamics and a unique and customized high-resolution simulator 704 for heart sound generation and propagation in a human thorax. The valve structural dynamics can be modeled according to the following equation:

$${m}_{p} = \frac{{d}^{2} \vec{x}}{d {t}^{2}} = Σ {\vec{F}}_{i n t} + Σ {\vec{F}}_{e x t} \qquad ( 1 )$$

with degrees of freedom on the order of 10⁵ and a typical CPU processing time in hours on the order of 10². The transvalvular hemodynamics can be modeled according to the following equations:

∇· [symbol] =0 (2)

$$\frac{\partial \vec{u}}{\partial t} + \vec{u} \cdot ∇ \vec{u} + \frac{∇ ρ}{ρ} = v {∇}^{2} \vec{u} \qquad ( 3 )$$

with degrees of freedom on the order of 10⁷ and a typical CPU processing time in hours on the order of 10⁴. The heart sound generation and scattering can be modeled according to the following equation:

$$ρ \frac{{\partial }^{2} {u}_{j}}{\partial {t}^{2}} - \frac{\partial }{\partial {x}_{j}} ( λ \frac{\partial {u}_{k}}{\partial {x}_{k}} {δ}_{ij} ) + \frac{\partial }{\partial {x}_{j}} [ μ ( \frac{\partial {u}_{j}}{\partial {x}_{j}} + \frac{\partial {u}_{j}}{\partial {x}_{j}} ) ] = {f}_{i} \qquad ( 4 )$$

with degrees of freedom on the order of 10⁷ and a typical CPU processing time in hours on the order of 10⁴.

For the valve dynamics computer modeling, a spring-network membrane model can be used, which can be coupled with a sharp-interface, immersed boundary based incompressible flow solver, such as ViCar3D, to resolve the blood flow dynamics through the valve. Vicar3D is a highly versatile, fully parallelized in-house immersed boundary solver that computes flow with complex moving/deforming bodies. The solver employs an efficient bi-conjugate gradient (BiCG) solver that scales well on up to about 1000 processors. The solver has been employed and validated for a wide range of studies of cardiac hemodynamics, including modeling of LV hemodynamics with natural and prosthetic mitral valves, and role of ventricular trabeculae on LV hemodynamics.

The sounds associated with the TAV can be generated using a Computational HemoAcousteomic (CHA) procedure where it has been shown that hemodynamic pressure fluctuations are the primary source of the heart sounds and the generated heart sound propagates through the inhomogeneous tissue medium in the form of compression as well as shear waves. A high-resolution, direct simulation method can be used for modeling wave propagation in tissue medium based on the immersed boundary, time domain finite-difference method, which has been validated against experimental measurements. The TAV sounds can be predicted by using this

CHA method. The hemodynamic pressure fluctuations obtained from the simulations of TAV hemodynamics can be used as a source term, and the heart sound propagation in real human thorax models can be performed by direct simulation of wave propagation.

The MTMS algorithm can be used as a forward model that can predict the measured signal in the array of sensors in the disclosed system. Hence, spatial-temporal sequences of patterns from the array of sensors can be used to estimate response from virtual sensors at any location on the body/thorax of the subject. In the disclosed physics “assisted” model-based machine intelligence and AI, MTMS data allows a learning algorithm to leverage data collected under one configuration to train the parameters for a novel configuration without collecting a new dataset. This approach also allows the development of personalized datasets for individuals tailored to the physical dimensions of the body, body anatomy as well as anatomy of internal structures (lung, heart dimensions etc.). Without the computational modeling-based machine intelligence approach disclosed herein, it would be increasingly difficult, if not impossible to harness the recent advances in deep learning with only the sensory data from the disclosed system because there would not be adequate pathological data to train robust neural network models.

Patient measurements, biomechanical models, and data-driven un-supervised learning techniques can be used to characterize the longitudinal acousteomic signatures of implanted TAVs. An understanding and modeling of the acousteomic signatures that correspond to the normal physiological effects of TAVs implant is a precursor to anomaly detection. Once an understanding and quantification of the acousteomic signal signature from “normal” TAVs is established, the signal of valve anomaly can be detected, such as leaflet thrombosis.

The most direct way of determining the various components of the “normal” acousteomic signature of TAVs is via a longitudinal auscultation study of patients. The first challenge in this task is that such a research study would have to dedicate tremendous resources to accomplish measurements at home for these patients, since the patients usually go home shortly after the procedure. The second challenge is that since murmurs associated with TAVs have not been analyzed to-date, there is not good insight as to what features/metrics provide the best detection of TAV malfunction.

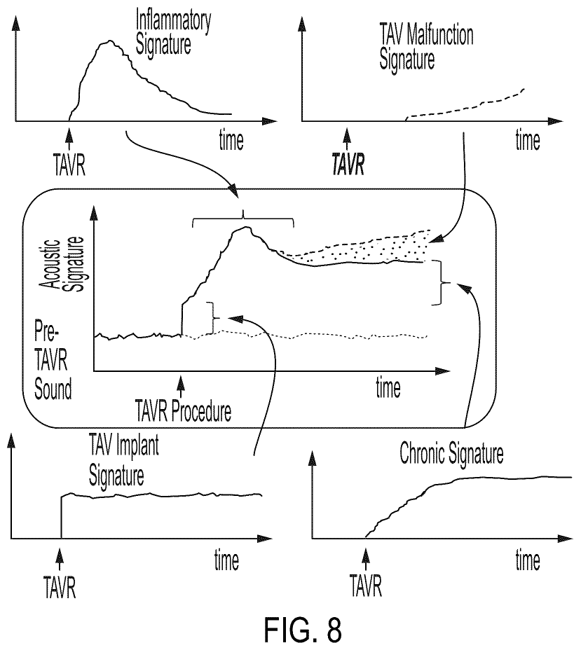

One approach to overcoming these challenges is based on the application of a machine-intellgence based statistical nonlinear regression which is informed by a combination of readily obtained, in-hospital patient measurements and comprehensive data from biophysical “in-silico” computational models of heart sounds. Based on the extensive TAVR experience of the clinical scientists as well as pilot data that has been gathered, the longitudinal acousteomic signature associated with an implanted TAV comprises three primary components as shown in FIG. 8. FIG. 8 shows hypothesized components of the acousteomic signature from TAV implanted in a patient based on cardiologist experience and our pilot data. Normal longitudinal acousteomic signature consists of “implantation”, “inflammatory”, and “chronic” signatures. The signature of TAV thrombosis is superposed on top of these “normal” signatures. The three primary component include the following: (1) an intrinsic signature of the TAV implant associated with its design and placement, (2) an acute, shorter term component associated with the initial inflammatory response to the procedure and its resolution, and (3) a longer term chronic signature associated with the adjustment of the patient's cardiac status to the implant such as changes in cardiac output, left-ventricle dilatation, stroke volume, aortic dilatation, etc. The signal of any TAV malfunction would overlay on the sum of these “normal” components. Mathematically, this can be expressed as:

F_{FAVR}(t)=P(t)+T(t)+I(t)+C(t)+M(t) (5)

where the components are described in Table 2 below where H(t) is the Heaviside function, and t₀ and t_{M} correspond to the times of the TAVR procedure and initiation of the malfunction.

Heart related signals are generated and propagate in a 3D space and evolve over time. Hence the spatiotemporal patterns of signals on the disclosed system correspond to the intertwined sequences of complex mechanical motions and flows in the cardiovascular system. To fully exploit the information from the disclosed sensor array, the following two data driven unsupervised learning techniques are employed.

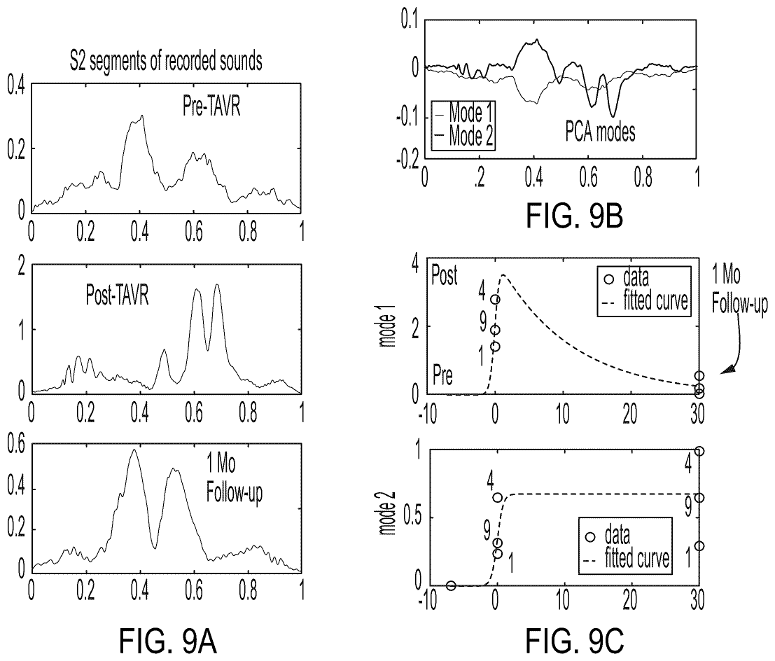

The first data driven unsupervised learning technique is a PCA like, linear sub-space projection that employs LDA (Linear Discriminant Analysis) and HLDA (Heteroscedastic LDA). FIGS. 9A-9C show pilot longitudinal measurement data from patients and PCA analysis to extract the acousteomic signatures. FIG. 9A shows this analysis for our pilot patient measurement data for the S2 sound at the pre-, post-TAVR, and 1-month follow-up. FIG. 9B shows modal shapes for the first 2 PCA modes of the signals, which contain about 90% of energy). FIG. 9C shows temporal variations of the modal amplitudes. Mode 1 and 2 correspond to the “inflammatory” and “implantation” signatures, presumed in FIG. 8 to be a decaying exponential and Heaviside respectively, with channel numbers noted beside the data points. FIGS. 9A-9C show that the temporal variation of the modal amplitudes represents the longitudinal change of the acousteomic signature. In a 4-patient dataset, it has been found that Modes 1 and 2 correlate with the “inflammatory” and “implantation” signatures, respectively. The curves fit to the regression models are also plotted in FIG. 9C. The data suggests that the inflammatory signal might subside significantly within a month of the procedure.

The second data driven unsupervised learning technique involves Delay Differential Analysis (DDA), a model based approach employed in the analysis of ECG data that creates an embedding of the multiple time series from the array of acousteomic sensors on the disclosed system into a multidimensional geometrical object. DDA enables the detection of frequencies, frequency couplings, and phases using nonlinear correlation functions and it is essentially a multivariate extension of discrete Fourier transform, for higher-order spectra. The two pattern analyses methods can be used to capture the intricate dynamics of TAV malfunction, its evolution in time and its manifestation in the regression model as shown in Table 2.

TABLE 2
Components of the acousteomic signal from a TAV
implanted in a patient based on cardiologist
intuition and corresponding candidate regression models.

| Acousteomic | | Candidate | Unknown |
|---|---|---|---|
| Signal | Expected | Regression | Param- |
| Component | Behavior | Model | eters |
| Pre-TAVR | Stable signature | P(t) = P₀[1 − H(t − t₀)] | P₀ |
| aortic | ceases after TAVR | | |
| valve sound | (t = t₀) | | |
| TAV implant | Stable signature that | T(t) = T₀[H(t − t₀)] | T₀ |
| sound | starts after TAVR | | |
| | (t = t₀) | | |
| Signal of | Initial rise post | I(t) = I₀[H(t − t₀)]. | I₀, I₁, |
| inflammatory response and its resolution | TAVR (t = t₀) followed by eventual decay | $[ \frac{{( t - {t}_{0} )}^{α}}{1 + {{I}_{1} ( t - {t}_{0} )}^{α + β}} ]$ | α(>0) B(>0), |
| Signal | Initial rise post | C(t) = C₀[H(t − t₀)]. | C₀, C₁, |
| associated with chronic | TAVR (t = t₀) followed by an | $[ \frac{{( t - {t}_{0} )}^{γ}}{1 + {{C}_{1} ( t - {t}_{0} )}^{γ}} ]$ | γ(>0) |
| response to | asymptotic state | | |
| TAVR | | | |
| Signal of a TAV | Initiated at time t = t_{W} | M(t) = | M′(t) |
| malfunction | and grows with time | [H(t − t_{M})]M′(t − t_{M}) | |

For the computer modeling, data from patients with “normal” TAV implant signatures can be partitioned randomly into two sets: one set for testing and one set for validation and tuning of the hyperparameters. This partitioning can be done repeatedly in a random fashion to enable cross-validation of the regression.

The in-silico MTMS model can be used to generate data on the longitudinal variation of the chronic signature of the TAV implant, i.e. the signature associated with the adjustment of the patient's cardiac status to the implant. Patients who receive a TAV implant usually experience a general improvement in their cardiac status including increase in cardiac output, reduction in blood pressure and reduction in aortic dilation. These chronic adjustments are reflected in the TAV sounds and MTMS can be used to quantify the effect of change in cardiac output (stroke volume) as well as reduction in aortic dilation on the TAV sounds. MTMS allows systematic evaluation of these effects via manipulation of the in-silico models, and complements the in-vivo, patient measurements.

The outcome of these studies can be used in a determination of the feature-set or metric(s) and the dimensionality of the heart sounds that can effectively characterize the longitudinal signature of a “normal” implanted TAV and a determination of the unknown parameters (see Table 2) in the candidate regression models. Regression model parameters can subsequently be employed as clinical markers in nonparametric Bayesian and statistical modeling techniques to predict dynamically failing trajectories and to address the challenge of individual vs population sources of variability.

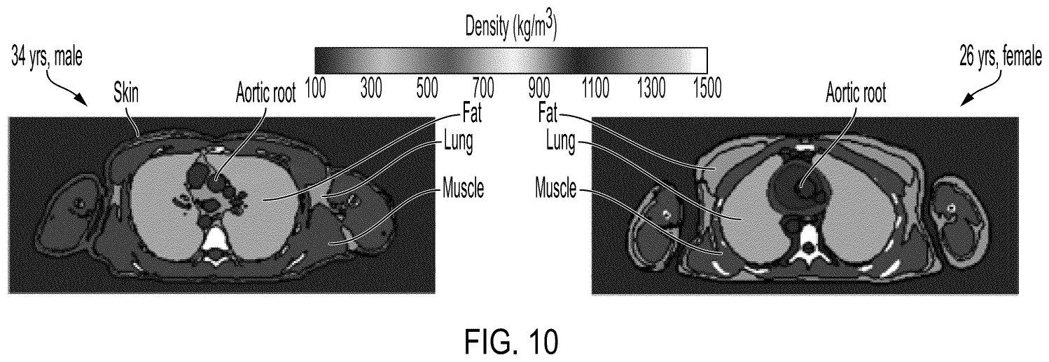

The presence of physician bias in patient-care associated with gender, race, body habitus and other factors is well documented. In principle, automated diagnosis via wearable sensors should reduce such bias. However, the design of the sensory device/system, algorithms as well as the intrinsic biophysics of the sensed signal introduce unique biases. For instance, blood pressure measurement via noninvasive sensors is less accurate for overweight patients. In the particular case of the disclosed system, the deformation waves associated with heart sounds propagate from the source (for instance, the TAV) to the precordium where it is measured, and these waves are affected by the chest wall thickness and the organs in the thorax. These deformation waves would undergo additional decay and diffraction in women (given the differences in male and female chest anatomy, as shown in FIG. 10, as well as in individuals with large body size, and/or high body mass index (BMI), thereby decreasing the signal-to-noise ratio (SNR). FIG. 10 shows male and female thorax anatomies derived from the ViP (Virtual Population) models. Note the significant differences in the two thoracic anatomies which will affect the heart murmur signal. Thus, careful consideration of these effects of body habitus as well as gender, will improve the diagnostic accuracy of the disclosed system.

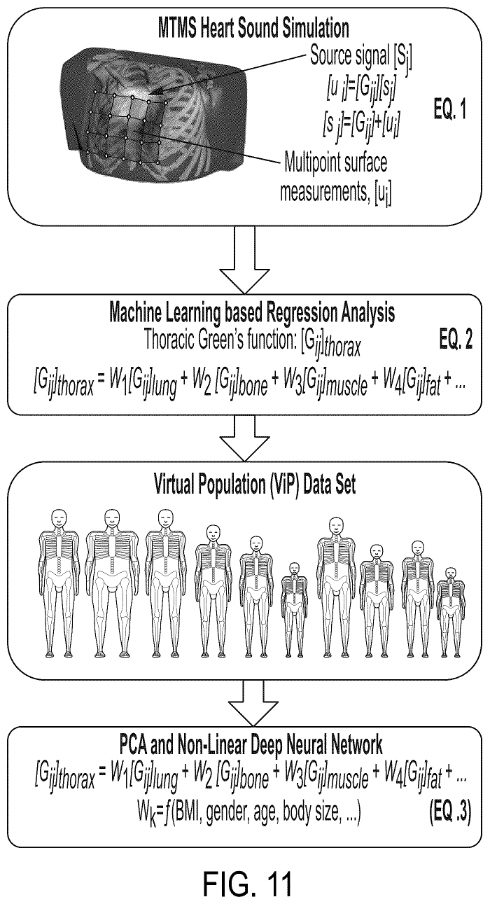

FIG. 11 shows a schematic scientific approach using modeling to determine bias due to body habitus and gender. Bioacousteomic factors associated with body habitus and gender represents a large parameter space, and the conventional approach to investigating these effects would be to conduct a large-cohort in-vivo patient study. However, such a study would be complex and expensive. Given the latter constraint, sufficient data is collected to generate a fundamental understanding regarding the effect of body habitus and gender on the heart sound signal sensed by the disclosed system, without employing an in-vivo study.

The in-silico study can use a high fidelity MTMS software tool to quantify and characterize the effect of body habitus and gender on the propagation of the heart sounds through the thorax and the sensed signal. For the computational study, high-resolution anatomical human models, Virtual Population 3.0 (ViP3.0) developed by IT'IS foundation, are used. The ViP3.0 includes 15 baseline male and female models (age 3-84 and BMI 13-36). The computational results based on adult male and female ViP3.0 models are used to develop a parameterized chest Green's function using principal component analysis (PCA).

As shown in FIG. 11, the HTMS heart sound simulation can be characterized by the following parameters: source signal [sⱼ], multipoint surface measurements [uⱼ], where

[uⱼ]=[G][sⱼ] (6)

[sⱼ]=[Gₛ][uⱼ] (7)

The machine intelligence-based regression analysis can be characterized by a Thoracic Green's function: [Gᵢⱼ]ₜₕₒᵣₐₓ and can be represented as follows:

[Gᵢⱼ]ₜₕₒᵣₐₓ=w₁[Gᵢⱼ]_{lung}+w₂[Gᵢⱼ]_{bone}+w₃[Gᵢⱼ]_{muscle}+w₄[Gᵢⱼ]_{fat}+ . . . (8)

The PCA and non-linear deep neural network can be represented as follows:

[Gᵢⱼ]ₜₕₒᵣₐₓ=w₁[Gᵢⱼ]_{lung}+w₂[Gᵢⱼ]_{bone}+w₃[Gᵢⱼ]_{muscle}+w₄[Gᵢⱼ]_{fat}+ . . . (9)

where wₖ=f(BMI, gender, age, body size, . . . )

Since heart sounds propagate through an inhomogeneous tissue medium, the measured heart sounds on the chest surface are affected by complex wave scattering due to different material properties and the geometry of the thorax, and this results in phase and amplitude differences in the sound measured on the chest surface. The relation between the signals at the measurement point, xi, and the source location, xj, can be described by a Green's function (see Eqs. 6 and 7). For a homogeneous viscoelastic medium, it has been shown that the Green's function can be obtained by using the free space Green's tensor, which is a function of the material properties of the medium, and the locations of source and measurement points. Once the Green's function is available, the multi-point measurement data, [ui] can be converted into the source signals, [sj] by using the pseudo inverse (see Eqs. 6 and 7) of the Green's function matrix and in doing so, the phase and amplitude modulation due to the wave propagation in the medium can be compensated. Hence, a method to evaluate the thoracic Green's function using the results from the MTMS based simulations is used. Since the source and measurement signals are already available from the MTMS simulations, the weight-factors, wk (Eq. 8 &9) are found by using machine-learning based regression models.

For a given body model, multiple simulations can be performed with various source signals and locations to improve the regression analysis. Moreover, the simulations can be performed without specific organs (e.g. without bone or lungs) to estimate the importance of each weighting factor. The Green's function evaluation can be applied to various body models in the ViP3.0 human model dataset. To expand the overall sample size, the ViP model morphing tool can be used to generate 8-10 additional models for each of the baseline adult models. The objective is to parameterize the weighting factors, wk for the primary parameters such as overall body size, BMI, and gender, using the principle components analysis (PCA) as well as non-linear methods of deep neural networks. Once this is accomplished, the approximated thoracic Green's function is obtained based on those primary parameters, and the thoracic Green's function can be used to process the measurement data obtained from the disclosed system.

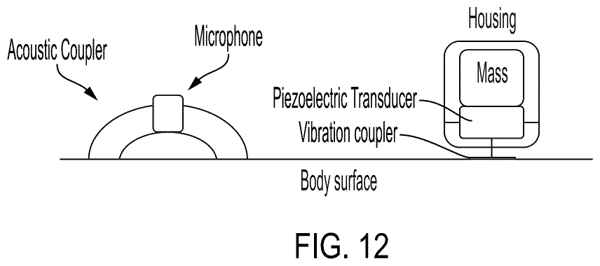

FIG. 12 shows two types of sensors for cardiac auscultation: acousteomic (left) and vibration (right). The results of the computational modeling can be used to inform the design of the disclosed system that can be personalized for an individual user, specifically in determining measurement locations that provide better informed signals for men and women of all body habitus. In addition to the placement of sensors, computational modeling of the body habitus canl be used to select particular type of sensor, as shown in FIG. 12. In one example of the disclosed system, Fukuda MA-250 sensors are used. In another example, Fukuda TH-306 sensor can be used. The features derived from the computational-based signal models can be used in conjunction with the gradient flow adaptive beamforming algorithm to combine the signals from sensors with the highest signal to noise ratio. Having optimal sensor location in conjunction with the gradient flow algorithm, localization can be improved, and thus effectively allowing the replacement of “the movement and placement of the stethoscope by the clinician's hand” with an all-electronic steering of data collection from the array of sensors.

By using this methodology, the Green's function associated with the propagation of the heart murmurs can be parameterized. This parameterization allows for the following; (a) enable a quantitative characterization of the effect of body habitus and gender (as parameterized by patient size, BMI, and other factors) on the propagation of heart sound signals; (b) determine those features of the measured signal which are minimally affected by body-habitus; and (c) provide insights into how the multiple simultaneous measurements can enable compensation for body habitus leading to quantitative and tractable ways to optimize the design of disclosed system and compare it with actual data that will be obtained from the clinical examination of the patients.

The virtual, in-silico virtual population models can be used to determine the effect of body habitus on heart murmur signals. The use of these virtual population models enables quantification of body habitus effects on heart murmurs in a way that is not possible via in-vivo studies. This modeling enables the generation of a comprehensive understanding of the effect of body habitus and the various thoracic organs on the heart murmur signals.

In some example, in-silico biomechanical models of thrombosed valves can be leveraged to augment patient measurement, thereby enabling the development of physics-based inference models, which can allow for robust detection and prediction of valve dysfunction.

For the regression model (Table 2), the signal associated with leaflet thrombosis is

M_{LT}(t)=[H(t−tₚ)]M′_{LT}(t−tₚ) (10)

where tₚ corresponds to the time of initiation of the malfunction, and M′_{LT} is the characteristic signature of leaflet thrombosis. Leaflet thrombosis primarily occurs in the sinus and/or on the sinus-facing side of the valve leaflets and consequently, thrombotic lesions initially affect the opening and closure of the affected leaflet(s). Thus, the initial acousteomic signature of leaflet thrombosis might appear in the earliest part of the systolic phase and of the second heart sound. Determination of these acousteomic features of TAV leaflet thrombosis is used to detect malfunction via automated auscultation.

While a seemingly straightforward way of extracting the signature of various TAV malfunctions is to conduct measurements of patients with these conditions, there are a number of challenges to this approach: (1) the absolute rate of incidence of these malfunctions is low (nominally about 10-15%), and therefore, obtaining sufficient samples of malfunctioning TAVs to train deep neural networks such as LSTM, is challenging; (2) in the current standard-of-care, most malfunction are usually detected at an advanced stage (due to appearance of symptoms) and thus, to obtain early signature of these malfunctions, one would have to continuously monitor a large number of TAVR recipients.

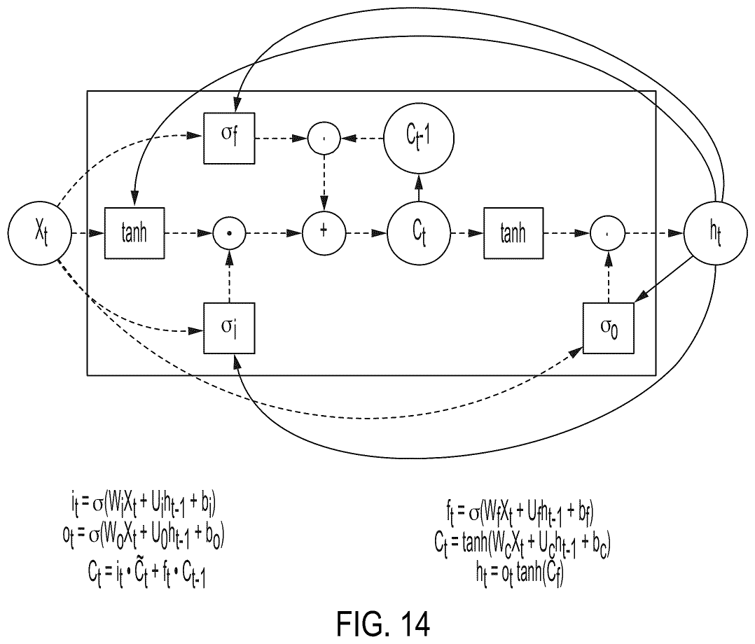

To help overcome the above challenges, the Multiphysics TAV Murmur Simulator (MTMS) is used for generating pathological sounds (N=100). This simulator allows for the ability to mimic and model the effect of leaflet thrombosis on measured heart sounds. The development of leaflet thrombosis and the associated leaflet thickening and Reduced Leaflet Motion (RLM) can be modeled with the disclosed fluid-structure interaction (FSI) valve model. In this model, the region of leaflet thrombosis is defined as schematically shown in FIGS. 14A-14C.

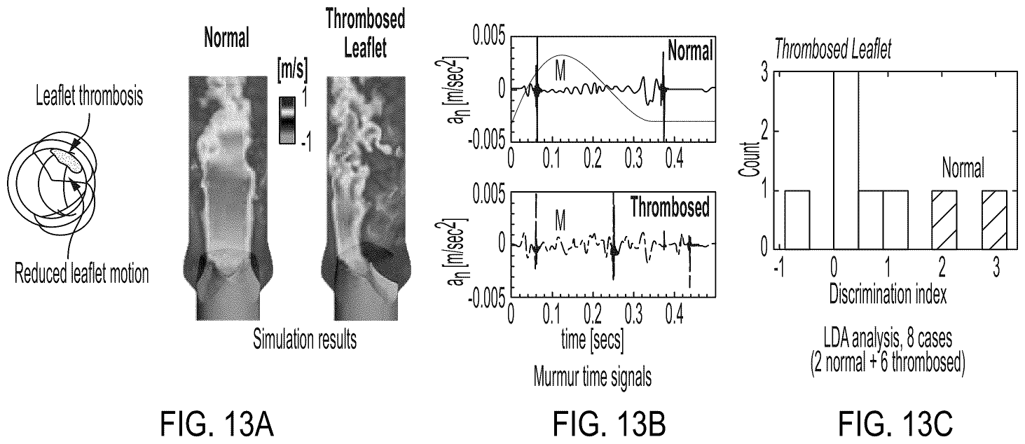

FIGS. 13A-13C shows modeling of leaflet thrombosis on valve sounds. Left: Schematic of leaflet thrombosis and resulting reduced leaflet motion. Right: Preliminary results from MTMS for a normal and leaflet thrombosis valves. FIG. 13A shows FSI simulation results showing the velocity contours at peak systole. FIG. 13B shows time signal of simulated heart sounds from normal and thrombosed valves. FIG.13C shows linear discriminant analysis for projection of PCA modes performed with 8 simulation cases for various thrombosis severities.

The structural elements affected by thrombosis are then identified, and the elastic stiffness (ke), point mass (mp), and effective bending modulus (Be) are increased for these elements based on the thickening of the leaflet. The elastic stiffness is directly proportional to the leaflet thickness, and the point mass and the effective bending modulus is computed by a linear combination based on the leaflet and thrombus thickness. Leaflet thrombosis and the resulting RLM can therefore be parametrically modeled by defining the region of thrombosis and the thrombus thickness.

FIGS. 13A-13C show pilot data (N=8) from MTMS simulations of heart sounds from normal and abnormal TAVs. FIG. 13A shows the FSI simulation results for a normal TAV and a TAV with thrombosis on one leaflet. The condition modeled here is subclinical since it corresponds to a transvalvular pressure gradient<5 mm Hg, which would not cause any obvious symptoms. This is exactly the kind of condition that we would like to proactively detect using automated auscultation. For the thrombosis case, one can clearly see that the aortic jet is deflected due to the incomplete opening of the stiffened leaflet. The TAV murmur simulation results are presented FIGS. 13A-13C and it is observed that, with leaflet thrombosis, the systolic murmur (marked by ‘M’) strength is increased, and the S2 sound is split into two parts. In the pilot data, we have performed 8 simulations with various degrees of leaflet thrombosis severity and carried out linear discrimination analysis (LDA) on the PCA modal projections of the various cases. As shown in FIG. 13C, the discrimination index obtained successfully discriminates normal from thrombosed valves.

In some examples, linear unsupervised learning methods such as PCA or LDA employed above can be useful. In other examples, advanced inference machine intelligence techniques that employ non-linear graphical models and deep learning may provide better results. However, given the low incidence rate of most pathologies (10-15% for leaflet thrombosis is this case) one concern associated with deep learning methods applied to detection of pathologies is the paucity of data, especially for cases with pathology, something that can be addressed using the MTMS simulations derived data. Simulations have demonstrated the power of in-silico models to augment the limited patient measurements of pathological valves. The power of in-silico modeling is that is allows for the systematic variability of the degree of leaflet thrombosis and development of a large, high-dimensional database for “learning” the acousteomic features that best identify this “malfunction.” Thus, the MTMS simulation (N˜100) can be combined with patient measurement of thrombosed valves (N˜20) to train the deep learning model.

While standard feedforward deep neural networks provide for a measure of statistical learning, the long short-term memory (LSTM) recurrent neural network architecture can be used as shown in FIG. 14, which is well-suited for learning complex dependencies across time such as those within the measurements obtained from the disclosed system from patients. FIG. 14 shows a schematic diagram of the LSTM node architecture is shown (top) with governing equations on the (bottom). The governing equations are as follows:

iₜ=σ(WᵢXₜ+Uᵢhₜ₋₁+bᵢ) (11)

oₜ=σ(WₒXₜ+Uₒhₜ₋₁+bₒ) (12)

Cₜ=iₜ·\tilde{C}ₜfₜ·Cₜ₋₁ (13)

fₜ=σ(W_{f}Xₜ+U_{f}hₜ₋₁+b_{f}) (14)

\tilde{C}ₜ=tanh (W_{C}Xₜ+U_{C}hₜ₋₁+b_{c}) (15)

hₜ=oₜ tanh (Cₜ) (16)

FIG. 14 detail the procedure to update each node at a given timestep. In these equations iₜ, fₜ, oₜ represent the value of the input, forget, and output gates respectively, \tilde{C}ₜ represents the update to the hidden state and Cₜ represents the current hidden state hₜ of a given node. Each node of the LSTM network maintains a hidden state that is updated at each timestep. In addition, each node contains an input, output, and forget gate, capable of controlling the behavior of the node depending on the current value of the hidden state. This architecture is thus capable of learning multiscale temporal dependencies in the data.

Signal patterns derived from over 100 individual MTMS simulations of TAVs with leaflet thrombosis as well as data collected using the disclosed system from a cohort of ˜20 patients with confirmed TAV thrombosis and ˜120 patients with normal valves can be used to train the deep neural network inference model. Knowledge derived from the MTMS simulations data can form the bulk of the deep neural network model and transfer learning can be employed to adapt the model to actual patient data. For patients with such malfunction, the standard-of-care requires evaluation via CT-scans and echocardiography of the valve. These provide data on the degree of leaflet dysfunction, and this data can also be used in the training of the ML algorithm. The training methodology uses physics as manifested in the MTMS data to “assist” the training of the deep neural network model. This approach addresses robustness, one of the key shortcomings of current data driven only approaches in ML and MI, a result of having models that have not been trained with adequate data. The deep neural network model can be used to discriminate the acousteomic signature of TAV thrombosis and can generate a fundamental understanding of the effect of these TAV malfunctions on hemodynamics, leaflet dynamics, TAV function and emitted sounds.

The disclosed systems and methods provides for a smart sensory system that allows for precise, personalized and robust physiological measurements from a multi-modality sensory array (acousteomic, vibration and electrical) using an embedded signal processing and cyber-physical systems as well as health based IoT for home health care. The disclosed systems and methods provide a distance health care delivery solution i.e. @Home, decongesting the hospitals for regular and postoperative patients. Moreover, the disclosed systems and methods can also be used in outpatient clinics, Cardiology Intensive Care (CIC) and Intensive Care Units (ICU) of hospitals for triaging patients using basic clinical personnel i.e. without the use of specialized trained medical staff. The disclosed systems and methods allow for detection and collection of high resolution spatio-temporal acousteomic sensing that can serve as a proactive early-warning system for the functioning of other cardiac prostheses (LVADs, embolization devices, grafts, shunts, stents.) as well as other surgical procedures (heart transplants etc.). Mapping other organ sounds can be employed to diagnose and monitor conditions of respiratory (asthma, lung collapse, sleep apnea), vascular (coronary artery disease, peripheral artery disease, aneurysms etc.) and the gastrointestinal system. Thus, the disclosed systems and methods provide for monitoring at home not only of cardiac prostheses but of respiratory, phonatory, orthopedic and other implants.

The heart sounds acquired using the disclosed systems can be modeled using one or more multi-physics models that couple flow, structural dynamics and acousteomics from first principles. A unified approach to sound generation and propagation is coupled with flow, leaflet dynamics and acousteomics modeling to provide unprecedented insights into heart sounds associated with valvular function, which can enable unique insights into the biophysics of this condition.

FIG. 15 is an example of a hardware configuration for an acousteomic processor 1500, which can be used to perform one or more of the processes described above. The acousteomic processor 1500 can be any type of acousteomic processors, such as desktops, laptops, servers, etc., or mobile devices, such as smart telephones, tablet computers, cellular telephones, personal digital assistants, etc. The acousteomic processor 1500 can be incorporated or formed as part of a remote monitoring system that is remote and/or separate from a patient and electrically coupled to a communications network (as described further below), a local monitory system that is in proximity to a patient, or both. As illustrated in FIG. 15, the acousteomic processor 1500 can include one or more processors 1502 of varying core configurations and clock frequencies. The acousteomic processor 1500 can also include one or more memory including compute in memory devices 1504 that serve as a main memory during the operation of the acousteomic processor 1500. For example, during operation, a copy of the software that supports the above-described operations can be stored in the one or more memory including compute in memory devices 1504. The acousteomic processor 1500 can also include one or more peripheral interfaces 1506, such as keyboards, mice, touchpads, computer screens, touchscreens, etc., for enabling human interaction with and manipulation of the acousteomic processor 1500.

The acousteomic processor 1500 can also include one or more network interfaces 1508 for communicating via one or more networks, such as Ethernet adapters, wireless transceivers, or serial network components, for communicating over wired or wireless media using protocols. The acousteomic processor 1500 can also include one or more storage devices 1510 of varying physical dimensions and storage capacities, such as flash drives, hard drives, random access memory, etc., for storing data, such as images, files, and program instructions for execution by the one or more processors 1502.

Additionally, the acousteomic processor 1500 can include one or more software programs 1512 that enable the functionality described above. The one or more software programs 1512 can include instructions that cause the one or more processors 1502 to perform the processes, functions, and operations described herein, for example, with respect to the process of described above. Copies of the one or more software programs 1512 can be stored in the one or more memory including compute in memory devices 1504 and/or on in the one or more storage devices 1510. Likewise, the data utilized by one or more software programs 1512 can be stored in the one or more memory including compute in memory devices 1504 and/or on in the one or more storage devices 1510. Peripheral interface 1506, one or more processors 1502, network interfaces 1508, one or more memory including compute in memory devices 1504, one or more software programs, and one or more storage devices 1510 communicate over bus 1514.

In implementations, the acousteomic processor 1500 can communicate with other devices via a network 1516. The other devices can be any types of devices as described above. The network 1516 can be any type of network, such as a local area network, a wide-area network, a virtual private network, the Internet, an intranet, an extranet, a public switched telephone network, an infrared network, a wireless network, and any combination thereof. The network 1516 can support communications using any of a variety of commercially-available protocols, such as TCP/IP, UDP, OSI, FTP, UPnP, NFS, CIFS, AppleTalk, and the like. The network 1516 can be, for example, a local area network, a wide-area network, a virtual private network, the Internet, an intranet, an extranet, a public switched telephone network, an infrared network, a wireless network, and any combination thereof.

The acousteomic processor 1500 can include a variety of data stores and other memory and storage media as discussed above. These can reside in a variety of locations, such as on a storage medium local to (and/or resident in) one or more of the computers or remote from any or all of the computers across the network. In some implementations, information can reside in a storage-area network (“SAN”) familiar to those skilled in the art. Similarly, any necessary files for performing the functions attributed to the computers, servers, or other network devices may be stored locally and/or remotely, as appropriate.

In implementations, the components of the acousteomic processor 1500 as described above need not be enclosed within a single enclosure or even located in close proximity to one another. Those skilled in the art will appreciate that the above-described componentry are examples only, as the acousteomic processor 1500 can include any type of hardware componentry, including any necessary accompanying firmware or software, for performing the disclosed implementations. The acousteomic processor 1500 can also be implemented in part or in whole by electronic circuit components or processors, such as application-specific integrated circuits (ASICs) or field-programmable gate arrays (FPGAs).

If implemented in software, the functions can be stored on or transmitted over a computer-readable medium as one or more instructions or code. Computer-readable media includes both tangible, non-transitory computer storage media and communication media including any medium that facilitates transfer of a computer program from one place to another. A storage media can be any available tangible, non-transitory media that can be accessed by a computer. By way of example, and not limitation, such tangible, non-transitory computer-readable media can comprise RAM, ROM, flash memory, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes CD, laser disc, optical disc, DVD, floppy disk and Blu-ray disc where disks usually reproduce data magnetically, while discs reproduce data optically with lasers. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Combinations of the above should also be included within the scope of computer-readable media.

The foregoing description is illustrative, and variations in configuration and implementation can occur to persons skilled in the art. For instance, the various illustrative logics, logical blocks, modules, and circuits described in connection with the embodiments disclosed herein can be implemented or performed with a general purpose processor, a digital signal processor (LINEAR ALGEBRA PROCESSOR (LAP)), an application specific integrated circuit (ASIC), a field programmable gate array (FPGA), cryptographic co-processor, or other programmable logic device, discrete gate or transistor logic, discrete hardware components, or any combination thereof designed to perform the functions described herein. A general-purpose processor can be a microprocessor, but, in the alternative, the processor can be any conventional processor, controller, microcontroller, or state machine. A processor can also be implemented as a combination of computing devices, e.g., a combination of a LINEAR ALGEBRA PROCESSOR (LAP) and a microprocessor, a plurality of microprocessors, one or more microprocessors in conjunction with a LINEAR ALGEBRA PROCESSOR (LAP) core, or any other such configuration.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

What is claimed is:

1. An appliance for monitoring blood flow comprising:
a plurality of spatially separated acousteomic sensors for auscultation detection of a patient;
a hardware processor and a non-transitory computer-readable medium that stores a trained computer model for modeling a function of a healthy heart for analyzing the acousteomic signals; and
a transmitter that transmits the acousteomic signals from the plurality of acousteomic sensors.

2. The appliance of claim 1, further comprising one or more electrocardiogram sensors that detect electrical signals produced by a heart.

3. The appliance of claim 2, wherein the trained computer model further analyzes the electrical signals.

4. The appliance of claim 5, wherein the trained computer model is trained using a physics-based virtual heart computer model that mimics the physical and physiological functioning of the heart.

5. The appliance of claim 1, wherein the analyzing comprises comparing the acousteomic signals from the plurality of acousteomic sensors with a baseline of known healthy acousteomic signals from the trained computer model.

6. The appliance of claim 3, wherein the analyzing comprises comparing the acousteomic signals from the plurality of acousteomic sensors and the electrical signals with a baseline of known healthy acousteomic signals and known healthy electrical signals from the trained computer model.

7. The appliance of claim 6, wherein the analyzing comprises determining an abnormality in at least one of the plurality of the acousteomic signals, at least one of the electrical signals, or both, based on the comparing.

8. The appliance of claim 7, wherein the abnormality comprises a thrombosis, a malfunction of an artificial valve, or both.

9. The appliance of claim 1, wherein the plurality of acousteomic sensors are part of a fabric that is physical contact with the patient.

10. A system for monitoring blood flow comprising:
a wearable garment comprising a plurality of spatially separated acousteomic sensors for auscultation detection of a patient and one or more electrocardiogram sensors that detect electrical signals produced by a heart of the patient;
a hardware processor and a non-transitory computer-readable medium that stores a trained computer model for modeling a function of a healthy heart for analyzing the acousteomic signals and the electrical signals; and
a transmitter that transmits the acousteomic signals and the electrical signals that are analyzed.

11. The system of claim 8, wherein the analyzing comprises comparing the acousteomic signals from the plurality of acousteomic sensors and the electrical signals with a baseline of known healthy acousteomic signals and known healthy electrical signals from the trained computer model.

12. The system of claim 8, wherein the analyzing comprises determining an abnormality in at least one of the plurality of the acousteomic signals, at least one of the electrical signals, or both, based on the comparing.

13. The system of claim 10, wherein the abnormality comprises a thrombosis, a malfunction of an artificial valve, or both.

14. The system of claim 8, wherein the trained computer model is trained using a physics-based virtual heart computer model that mimics the physical and physiological functioning of the heart.

15. A computer-implemented method for monitoring blood flow comprising:
detecting auscultation using a plurality of spatially separated acousteomic sensors for a patient;
analyzing the acousteomic sensors using a hardware processor and a non-transitory computer-readable medium that stores a trained computer model for modeling the function of a healthy heart; and
transmitting the acousteomic signals from the plurality of acousteomic sensors.

16. The computer-implemented method of claim 15, further comprising detecting electrical signals using one or more electrocardiogram sensors that detect electrical signals produced by a heart.

17. The computer-implemented method of claim 16, wherein the trained computer model further analyzes the electrical signals.

18. The computer-implemented method of claim 17, wherein the trained computer model is trained using a physics-based virtual heart computer model that mimics the physical and physiological functioning of the heart.
